(19) United States
(12) Patent Application Publication
(10) Pub. No.: US 2025/0326940 A1
(43) Pub. Date: Oct. 23, 2025

(54) HYDROPHILIC COATINGS FOR VASCULAR MEDICAL PRODUCTS

(71) Applicant: BIOTRONIK AG, Buelach (CH)

(72) Inventors: Alwin Schwitzer, Buelach (CH); Martin Winkler, Galgenen (CH); Konstantin Siegmann, Greifensee (CH); Linyu Mu, Lemfoerde (CH); Jan Inauen, Uster (CH)

(21) Appl. No.: 18/861,924

(22) PCT Filed: May 23, 2023
(86) PCT No.: PCT/EP2023/063740

§ 371 Date: Oct. 31, 2024

(30) Foreign Application Priority Data
May 25, 2022 (EP) 22175573.9
Apr. 13, 2023 (EP) 23167774.1

(51) Int. Cl. C09D 5/16; A61L 29/08; C08F 226/10; C08J 7/04; C09D 7/20; C09D 139/06

(52) U.S. Cl. CPC C09D 5/1668; A61L 29/085; C08F 226/10; C08J 7/0427; C09D 7/20; C09D 139/06; A61L 2420/02; C08F 2800/10; C08J 2339/06

(57) ABSTRACT

A hydrophilic coating for coating a surface of a vascular medical product. The copolymer includes repeating units of (a) vinylpyrrolidone, and (b) perfluorophenylazide. The copolymer can have a statistical distribution of the repeating units (a) and (b) and a molecular weight of 5,000 to 50,000 g/mol. The coating can be applied and then dried or radiated with UV radiation.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2023/063740, which was filed May 23, 2023, which application claimed priority from EP Application Serial No. 22175573.9, which was filed May 25, 2022 and from EP Application Serial No. 23167774.1, which was filed Apr. 13, 2023.

### FIELD OF THE INVENTION

A field of the invention is hydrophilic coatings for surfaces of vascular medical products.

### BACKGROUND

It is generally known to coat vascular medical products, such as catheters, balloon parts, or pacemaker electrodes, with hydrophilic polymers. Vascular medical devices require a lubricious coating to facilitate insertion into blood vessels and minimize damage to them. Lubrication is achieved by a hydrophilic coating that absorbs water and thus becomes slippery. However, the known systems are either expensive due to the chemicals used, or do not reliably meet the requirements for vascular implantable medical products, particularly providing a permanent coating on the surfaces with very low number of particles released, and/or low friction to blood vessels. It is further known that hydrophobic surfaces, such as polyolefins or polysiloxanes, are difficult to coat with hydrophilic compounds.

### SUMMARY OF THE INVENTION

A preferred coating for surface of a vascular medical product or at least one part thereof includes or consists of repeating units of a) vinylpyrrolidone and b) at least one perfluorophenyl azide moiety. The coating can be applied by dip coating, spray coating, roller coating, printing, painting, or application with a doctor blade, doctor roller or Langmuir-Blodgett films. The coating can be dried or irradiated with UV radiation after application.

### BRIEF DESCRIPTION OF THE DRAWINGS

Embodiments of the invention shall be described in detail with reference to the drawings. It is to be noted that the embodiments are not limiting for the invention, but merely represent illustrative examples. The figures show:

FIG. 1 shows a procedure for adding the perfluorophenyl (meth)acrylate into the polymerization vessel via a syringe pump;

FIG. 2 shows the molecular weight distribution of an exemplary copolymer by GPC;

FIG. 3 shows the 19F-NMR spectrum of an exemplary copolymer;

FIG. 4 shows the 1H-NMR spectrum of an exemplary copolymer;

FIG. 5 shows 1H-NMR, 19F-NMR and FT-IR spectra as well as molecular weight distribution for another exemplary copolymer; and

FIG. 6 shows schematically the binding of the copolymer to a surface.

### DETAILED DESCRIPTION OF THE PREFERRED EMBODIMENTS

The invention concerns hydrophilic copolymers that can be covalently bound to the surfaces of vascular medical products. Preferred coatings are hydrophilic and allow an easy to perform or automatic application. The coatings are applicable to surfaces that are difficult to coat, e.g., hydrophobic surfaces. The coatings provide low friction to friction partners, e.g., blood vessels, and release of a very low number of particles.

Preferred coatings and methods covalently bind the coating by drying or by application of UV-radiation. Preferred coating compositions include or consists at least one of the following copolymers having at least
a) repeating units of vinylpyrrolidone, and
b) at least one perfluorophenyl azide moiety, and optionally the applied copolymeric coating is dried and/or irradiated by UV irradiation.

Thus, a method for coating a surface of a vascular medical product or at least one part thereof with a copolymeric coating according to the following chemical process is described:

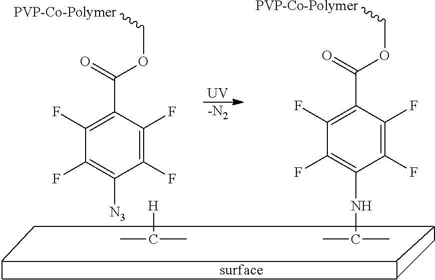

wherein the at least one copolymer includes
a) repeating units of vinylpyrrolidone, and
b) at least one perfluorophenyl azide moiety,
wherein the at least one perfluorophenyl azide moiety is inserted in a chemical C—H bond of the surface of the vascular medical product (e.g. an interventional catheter) or the at least one part thereof, preferably by irradiating the copolymeric coating and the surface of the vascular medical product or the at least one part thereof with UV irradiation.

The copolymeric coating can be applied to the surface of the vascular medical product or to the at least one part thereof by dip coating, spray coating, roller coating, printing, painting, or by means of a doctor blade, doctor roller, or Langmuir-Blodgett films, then the applied composition is dried and irradiated by UV light. The surface of the vascular medical product or the at least one part thereof may be dipped into the coating composition and drawn out from the coating composition at a defined velocity to obtain a defined coating thickness. The method may be carried out in automated manner.

The surface of the vascular medical product or the at least one part thereof may include a polymer selected from a thermoplastic elastomer, polyamide, polyetherblockamides, thermoplastic polyurethanes (TPU), polyester, polyester-based elastomers, polyolefins, vinylpolymers, polysiloxanes, and combinations thereof, preferably selected from a thermoplastic elastomer, a polyamide, a polyetherblockamide, thermoplastic polyurethanes (TPU), thermoplastic elastomer (TPE) such as a thermoplastic elastomer including polyamide and polyether blocks, preferably a polyether block amide (e.g. Pebax or Vestamid).

Thus, the obtained vascular medical product is coated with a (hydrophilic) copolymeric coating including at least one of the following copolymers or consisting of the following copolymer:

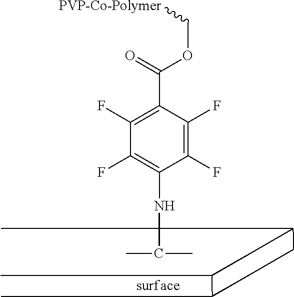

Therefore, a vascular medical product or at least one part thereof including a copolymeric coating is described, wherein the copolymeric coating is bound to a surface of the vascular medical product and includes at least one of the following copolymers or consists of the following copolymer:

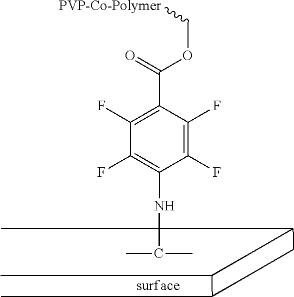

The above described (hydrophilic) copolymeric coating that absorbs water and thus becomes slippery enables a lubrication within the human or animal body (e.g. in a blood vessel). The (hydrophilic) copolymeric coating is mechanically resistant. The (hydrophilic) copolymeric coating is chemically bound to the surface of the vascular medical product or the at least one part thereof. Thus, no particles are released from the surface, therefore the (hydrophilic) copolymeric coating poses a lower risk for thrombosis, embolism, and arterial blockage. The (hydrophilic) copolymeric coating show very good sliding and abrasion resistance compared to prior art products.

Water-soluble polyvinylpyrrolidone (PVP) serves for the hydrophilic properties. Mono- as well as multi-functional perfluorophenyl azide (PFPA) copolymers can be used for crosslinking and chemical bonding to the vascular medical product or the part thereof, e.g. an (interventional) catheter surface.

In one embodiment the vascular medical product may include a (hydrophilic) copolymeric coating, wherein the (hydrophilic) copolymeric coating includes or consists of the following copolymer:

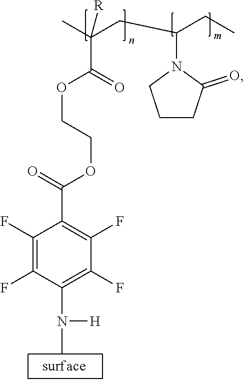

wherein R is H or CH3 and wherein n is an integer 1 or 2, and m is an integer in the range of from 15 to 35, preferably wherein n is an integer 1 and m is an integer in the range of from 20 to 30.

In another embodiment the vascular medical product may include a (hydrophilic) copolymeric coating, wherein the (hydrophilic) copolymeric coating includes or consists of the following copolymer:

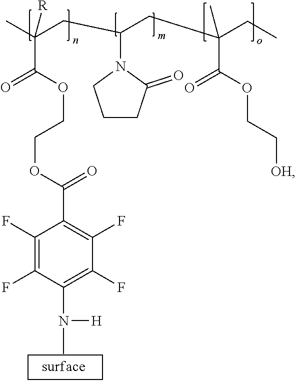

wherein R is H or CH3 and wherein n is 1 or 2, m is 15 to 25, and o is 5 to 15, preferably wherein n is 1, m is 20, and o is 10.

In a further embodiment the vascular medical product may include a (hydrophilic) copolymeric coating, wherein the (hydrophilic) copolymeric coating includes or consists of the following copolymer:

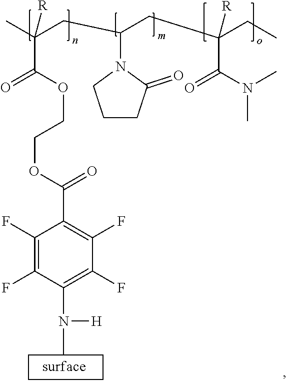

wherein R is H or CH3 and wherein n is 1 or 2, m is 15 to 25, and o is 5 to 15, preferably wherein n is 1, m is 20, and o is 1 to 10.

The (hydrophilic) copolymeric coating may be (covalently) bound to a surface of the vascular medical product, preferably via a carbon nitrogen (C—N) bond.

The vascular medical product is a medical product or a part thereof, which is intended to be implanted or introduced into a vessel, particularly in the fields of electrophysiology, heart-rhythm management, neurostimulation, vascular and endovascular intervention, intervention in and at the heart. The vascular medical product may be selected from coronary or peripheral catheters or parts thereof, CRM devices or parts, balloon parts, or pacemaker electrodes. The surface of the vascular medical product may include or may be made of a polymer selected from a thermoplastic elastomer, polyamide, polyetherblockamides, thermoplastic polyurethanes (TPU), polyester, polyester-based elastomers, polyolefins, vinylpolymers, polysiloxanes, and combinations thereof, preferably selected from a, a polyamide, a polyetherblockamide, thermoplastic polyurethanes (TPU), thermoplastic elastomer (TPE) such as a thermoplastic elastomer including polyamide and polyether blocks, preferably a polyether block amide (e.g. Pebax or Vestamid).

The (hydrophilic) copolymeric coating can be applied to the vascular medical product or a part thereof, e.g. (interventional) catheter (hose), via a dip coating process.

Also a vascular medical product, including a (hydrophilic) copolymeric coating is disclosed wherein the (hydrophilic) copolymeric coating includes or consists of the following segments:
an azide,
an aromatic moiety being bound to the azide,
an ester being bound to the aromatic moiety,
at least one hydrophilic repeating unit being bound to the ester.

In one embodiment the vascular medical product includes a (hydrophilic) copolymeric coating wherein the (hydrophilic) copolymeric coating includes or consists of the following segments:
an azide,
a perfluorophenyl moiety being bound to the azide,
an ester being bound to the perfluorophenyl moiety,
an acrylate or methacrylat or repeating units of an acrylate or methacrylate being bound to the ester,
repeating units of vinylpyrrolidone being bound to the acrylate or methacrylat or repeating units of an acrylate or methacrylate, and optionally
repeating units being bound to the vinylpyrrolidone repeating units and wherein the repeating units are selected from of 2-hydroxyethyl methacrylate (HEMA), N,N-dimethyl (meth)acrylate and N,N-dimethylacrylamide (DMA).

Due to its hydrophobic nature the phenylazide can be bound preferably to hydrophobic surfaces, e.g. that of a vascular medical product. The aromatic moiety, preferably the perfluorophenyl moiety, is configured to absorb UV irradiation and to activate the azide. The azide is a reactive group, which upon activation, preferably UV-irradiation, splits off dinitrogen (N₂) forming a highly reactive nitrene intermediate that is able to insert into a C—H bond of a vascular medical product surface. Thus, the azide or the copolymer including an azide group becomes covalently bond to the surface of the vascular medical product. The (poly) vinylpyrrolidone provides hydrophilic properties.

Furthermore, the present invention meets this need at least in part by providing use of a UV-sensitive copolymer for coating a surface of a vascular medical product, wherein the UV-sensitive copolymer includes at least repeating units of (a) vinylpyrrolidone, and (b) perfluorophenylazide, wherein the copolymer has a statistical distribution of the repeating units (a) and (b) and has a molecular weight of 5,000 to 50,000 g/mol. The invention further relates to use of a coating composition for coating a surface of a vascular medical product, wherein the coating composition contains the copolymer, a method for coating the surface of a vascular medical product, and the coated vascular medical products obtained.

The vascular medical product may relate to a medical product or a part thereof which may be or are (to be) inserted or introduced into a vessel, e.g. but not limited in the fields of electro-physiology, cardiac rhythm management, neurostimulation, vascular or endovascular intervention as well as cardiac intervention.

The surface of the vascular medical product includes a polymer selected from a thermoplastic elastomer (TPE), polyamide, polyetherblockamides, thermoplastic polyurethanes (TPU), polyester, polyester-based elastomers, polyolefins, vinylpolymers, polysiloxanes, and combinations thereof. The surface of the vascular medical product preferably includes a polymer selected from a thermoplastic elastomer, a polyamide, a polyetherblockamide, thermoplastic polyurethanes (TPU), thermoplastic elastomer (TPE) such as a thermoplastic elastomer including polyamide and polyether blocks, preferably a polyether block amide (e.g. Pebax or Vestamid). A polyether block amide is a block copolymer obtained by polycondensation of a carboxylic acid polyamide (PA6, PA11, or PA12) with an alcohol termination polyether (Polytetramethylene glycol (PTMG) or PEG).

Herein, the term “(meth)acrylate” is used to designate “acrylate” or “methacrylate”, as commonly used in the art.

Herein, the term “slippery” in the context of a surface of a vascular medical product has the meaning that the vascular medical product can be inserted into a body without leading to injuries and/or causing pain or discomfort to the patient.

Further examples of the present disclosure are provided below:

Example 1. A vascular medical product, including a (hydrophilic) copolymeric coating, wherein the (hydrophilic) copolymeric coating includes or consists of a copolymer having the following segments:
an azide,
an aromatic moiety being bound to the azide,
an ester being bound to the aromatic moiety,
at least one hydrophilic repeating unit being bound to the ester.

Example 2. A vascular medical product according to example 1, including a (hydrophilic) copolymeric coating, wherein the (hydrophilic) copolymer includes the following segments:
an azide,
an aromatic moiety being bound to the azide,
an ester being bound to the aromatic moiety,
an acrylate or methacrylat or repeating units of an acrylate or methacrylate being bound to the ester,
repeating units of vinylpyrrolidone being bound to the acrylate or methacrylat or repeating units of an acrylate or methacrylate, and optionally
repeating units being bound to the vinylpyrrolidone repeating units and wherein the repeating units are selected from of 2-hydroxyethyl methacrylate (HEMA), N,N-dimethyl (meth)acrylate and N,N-dimethylacrylamide (DMA).

Example 3. A vascular medical product, including a (hydrophilic) coating, wherein the (hydrophilic) coating includes at least one copolymer having at least
a) repeating units of vinylpyrrolidone, and
b) at least one perfluorophenyl azide moiety.

Example 4. The vascular medical product according to example 3, wherein a repeating unit of vinylpyrrolidone includes more monomer units than a repeating unit of perfluorophenyl azide moiety.

Example 5. The vascular medical product according to example 3 or 4, wherein the copolymer includes 1 to 50 mol % of the repeating unit a), and 0.1 to 5 mol % of the repeating unit b).

Example 6. The vascular medical product according to one of the examples 3 to 5, wherein the copolymer has a statistical distribution of the repeating units a) and b).

Example 7. The vascular medical product according to any one of the examples 3 to 6, wherein the copolymer further includes at least one repeating unit c) selected from of 2-hydroxyethyl methacrylate (HEMA), N,N-dimethyl (meth)acrylate, and N,N-dimethylacrylamide (DMA).

Example 8. The vascular medical product according to example 7, wherein the copolymer includes 1 to 20 mol % of the repeating unit c), preferably in a statistical distribution.

Example 9. The vascular medical product according to any one of the preceding examples, wherein the copolymer has a molecular weight of 5,000 to 50,000 g/mol.

Example 10. The vascular medical product according to any one of the examples, wherein the (hydrophilic) copolymeric coating is (covalently) bound to a surface of the vascular medical product, preferably via a carbon nitrogen (C—N) bond.

Example 11. The vascular medical product according to any one of the examples, wherein the vascular medical product is a medical product or part thereof, which is intended to be implanted or introduced into a vessel, particularly in the fields of electrophysiology, heart-rhythm management, neurostimulation, vascular and endovascular intervention, intervention in and at the heart.

Example 12. The vascular medical product according to any one of the examples, wherein the vascular medical product is selected from coronary or peripheral catheters or parts thereof, CRM devices or parts, balloon parts, or pacemaker electrodes.

Example 13. The vascular medical product according to any one of the examples, wherein the surface of the vascular medical product includes or is made of a polymer selected from a thermoplastic elastomer, polyamide, polyetherblockamides, thermoplastic polyurethanes (TPU), polyester, polyester-based elastomers, polyolefins, vinylpolymers, polysiloxanes, and combinations thereof, preferably selected from a, a polyamide, a polyetherblockamide, thermoplastic polyurethanes (TPU), thermoplastic elastomer (TPE) such as a thermoplastic elastomer including polyamide and polyether blocks, preferably a polyether block amide (e.g. Pebax or Vestamid).

Example 14. Use of a copolymeric coating for coating a surface of a vascular medical product, wherein the copolymeric coating includes or consists of a copolymer having at least
a) repeating units of vinylpyrrolidone, and
b) at least one perfluorophenyl azide moiety, and optionally
c) repeating units selected from of 2-hydroxyethyl methacrylate, N,N-dimethyl (meth)acrylate, and N,N-dimethylacrylamide.

Example 15. Use of a (UV-sensitive, hydrophilic) copolymer for coating a surface of a vascular medical product, wherein the (UV-sensitive, hydrophilic) copolymer includes at least repeating units of
(a) vinylpyrrolidone, and
(b) perfluorophenylazide,
wherein a repeating unit of vinylpyrrolidone includes more monomer units than a repeating unit of perfluorophenylazide;
wherein the copolymer has a statistical distribution of the repeating units (a) and (b) and has a molecular weight of 5,000 to 50,000 g/mol.

Example 16. The use according to example 14 or 15, wherein the copolymer includes 1 to 50 mol % of the repeating unit (a), and 0.1 to 5 mol % of the repeating unit (b).

Example 17. The use according to example 14, wherein the copolymer includes 1 to 20 mol % of the repeating unit (c), preferably in a statistical distribution.

Example 18. Use of a (UV-sensitive, hydrophilic) copolymer for coating a surface of a vascular medical product, wherein the copolymer includes the following repeating units:

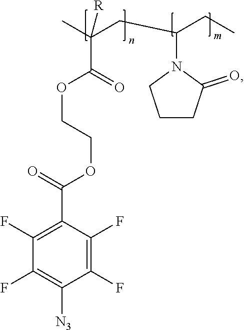

wherein n is an integer 1 or 2, m is an integer in the range of from 15 to 30 and wherein R is H or CH3.

Example 19. Use of a (UV-sensitive, hydrophilic) copolymer for coating a surface of a vascular medical product, wherein the copolymer includes the following repeating units:

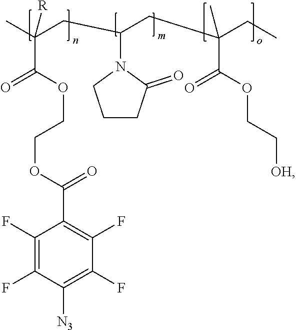

wherein n is 1, m is 20, and o is 10 and wherein R is H or CH3; or

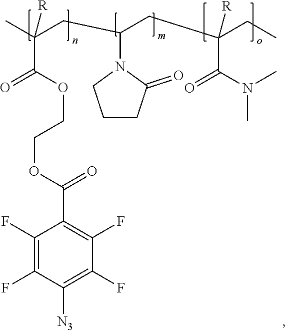

wherein n is 1, m is 20, and o is 10 and wherein R is H or CH3.

Example 20. Use of a coating composition for coating a surface of a vascular medical product, wherein the coating composition includes:
a) 0.1 to 10 weight % of polyvinylpyrrolidone having a molecular weight of less than 1,000,000 g/mol,
b) 0.1 to 10 weight % of polyvinylpyrrolidone having a molecular weight of more than 1,000,000 g/mol,
c) 0.1 to 5 weight % of a copolymer as defined in any one of the preceding claims examples, and
d) 0 to 1 weight % of a multifunctional UV-sensitive lower-molecular phenyl azide compound having an UV-absorption in the range of 200 to 300 nm,
wherein the components (a) to (d) are dissolved in 50 to 90 volume % of alcohol, preferably ethanol and/or 2-propanol, and 10 to 50 volume % of water.

Example 21. The use according to example 20, wherein the phenyl azide compound d) is a triazide, polyethyleneglycol-based bisazide, diol bisazide, and/or an ionic bisazide.

The copolymer used according to the invention includes at least repeating units of vinylpyrrolidone (VP), and repeating units of perfluorophenylazide (PFPA), preferably perfluorobenzoyl (meth)acrylate azide. A repeating unit of vinylpyrrolidone includes more monomer units than a repeating unit of perfluorophenylazide includes monomer (PFPA) units. Preferably, a repeating unit of vinylpyrrolidone (VP) includes 15 to 35 VP units, more preferably 20 to 30 VP units. Preferably, a repeating unit of PFPA includes 1 to 3 PFPA units, more preferably 1 or 2 PFPA units, most preferably 1 PFPA unit. An exemplary copolymer includes the following repeating units:

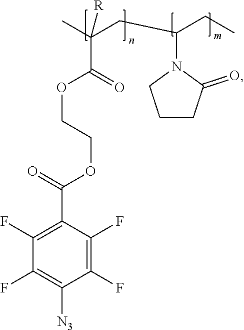

wherein n is an integer of 1, 2 or 3, preferably 1 or 2, and wherein m is an integer in the range of from 15 to 35 and wherein R is H or CH3.

The polyvinylpyrrolidone repeating units impart hydrophilic and slippery properties to the coatings obtained by using the copolymer. The perfluorophenylazide units enable to covalently bind or attach the copolymer to surfaces, particularly to polymeric or siloxane materials, via the azide group which can be activated by UV-radiation. The reaction mechanism of the activation by the azide group is known from the article of Siegmann, K., Inauen, J., Villamaina, D. and Winkler, M., “Photografting of perfluoroalkanes onto polyethylene surfaces via azide/nitrene chemistry.” Applied Surface Science, 396 672-680 (2017), and the article of Siegmann, K., Inauen, J., Sterchi, R. and Winkler, M., “Spectroscopy on photografted polyethylene surfaces using a perfluorophenyl azide: Evidence for covalent attachment.” Surface and Interface Analysis, 50 (2) 205-211 (2018). The mechanism is shown hereinafter, wherein R designates the corresponding residue of the perfluorophenylazide unit:

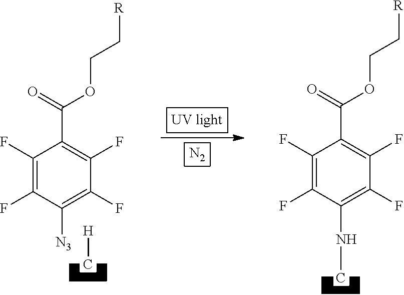

Upon UV activation, preferably at a wavelength between 200 and 350 nm, more preferably between 240 and 320 nm, the azide group is converted to molecular nitrogen which splits off, and a highly reactive nitrene, which undergoes a very fast non-specific insertion reaction into a C—X bond of a substrate, for example, a C—H bond. Due to the presence of several PFPA repeating groups and thus several azide groups in a copolymer, the copolymer can be covalently bound and thus permanently attached at these several positions to the substrate.

The copolymer has a statistical distribution of the VP repeating units (a) and PFPA repeating units (b) and has a molecular weight of 5,000 to 50,000 g/mol, preferably of 10,000 to 45,000 g/mol.

In preferred embodiments, the copolymer includes further hydrophilic repeating units, for example, a repeating unit (c) derived from 2-hydroxyethyl methacrylate (HEMA), N,N-dimethyl (meth)acrylate, and N,N-dimethylacrylamide (DMA). The additional repeating units further improve the hydrophilic and slippery performance of coatings obtained by using the copolymer.

An exemplary copolymer can include the following repeating units:

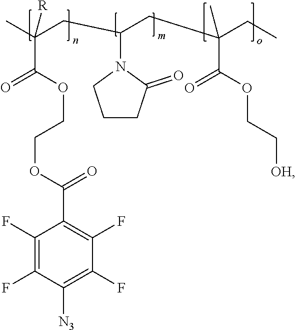

wherein, for example, n is 1, m is 15 to 25, preferably 20, and o is 5 to 15, preferably 10; and wherein R is H or CH₃.

Another exemplary copolymer can include the following repeating units:

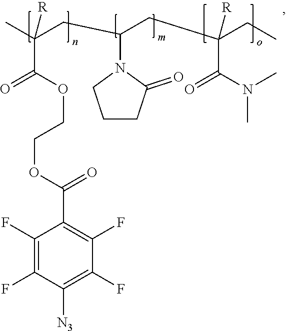

wherein, for example, n is 1, m is 15 to 25, preferably 20, and o is 5 to 15, preferably 10, and wherein R is H or CH₃.

In preferred embodiments, the copolymer used according to the invention includes 1 to 50 mol % of vinylpyrrolidone repeating unit (a), preferably 5 to 45 mol % of vinylpyrrolidone repeating unit (a), and 0.1 to 5 mol % of PFPA repeating unit (b), preferably 1 to 5 mol % of PFPA repeating unit (b). In exemplary copolymers that include further repeating units (c) as described above, the copolymer includes 1 to 20 mol % of the repeating unit (c). In further preferred embodiments, the repeating units (a), (b) and (c) are statistically distributed.

The copolymer used according to the invention can be prepared by a process including at least the following steps:
(a) esterification of perfluorobenzoyl chloride with 2-hydroxyethyl (meth)acrylate;
(b) reacting the product obtained in step (a) with sodium azide, to obtain 4-azidoperfluorophenyl acrylate; and
(c) copolymerizing the 4-azidoperfluorophenyl azide acrylate of step (b) with vinylpyrrolidone using a radical initiator.

The esterification step (a) can be carried out as described in the following reaction scheme:

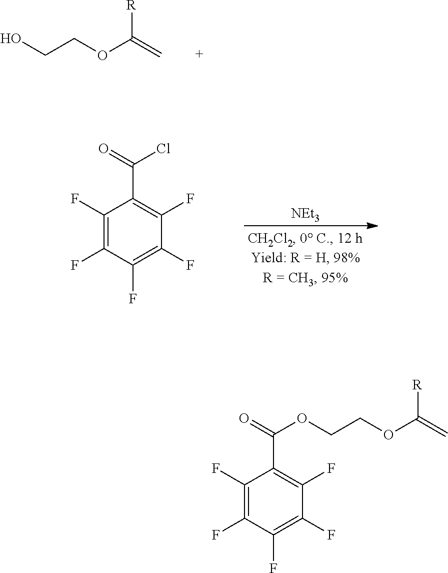

As shown, perfluorobenzoyl chloride is reacted with a 2-hydroxyethyl (meth)acrylate with R designating H or methyl, in triethylamine. In exemplary embodiments, the reaction is carried out in dichloromethane at about 0° C. and for about 12 hours. Exemplary yields are, 98% for R═H, and 95% for R=methyl. The compounds obtained can be characterized by e.g., FT-IR, 1H and 19F-NMR spectroscopy.

Compounds preferably obtained after step (a) are shown hereinafter (left: methacrylate; right: acrylate):

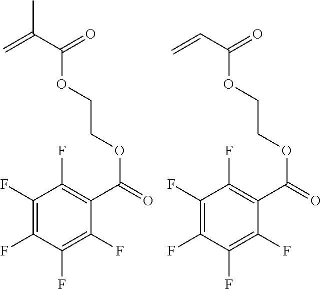

It is also possible to use an acrylamide or vinylether, instead of 2-hydroxyethyl (meth)acrylate, resulting in the following compounds (left: acrylamide; right: vinylether):

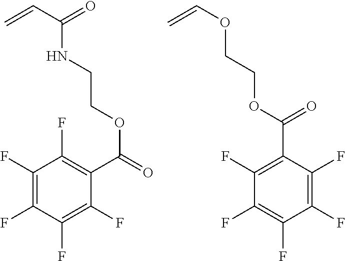 [SMILES: C=CC(=O)NCCOC(=O)c1c(F)c(F)c(F)c(F)c1F ; C=COCCOC(=O)c1c(F)c(F)c(F)c(F)c1F]

After step (a), the product is reacted with an azide, preferably sodium azide. The reaction can be carried out under suitable conditions, e.g., at room temperature and for 24 hours. In step (b), the azide group is introduced by substituting a F atom of the perfluorophenyl ring.

In step (c), the product obtained in step (b) is polymerized with vinylpyrrolidone, wherein vinylpyrrolidone is used in excess of the azidoperfluorophenyl acrylate. The copolymerization is initiated with a radical polymerization initiator, for example, AIBN or dibenzoyl peroxide (BPO). The copolymerization is carried out in a manner known in the art.

An exemplary method with the steps (a) to (c) is shown in the scheme hereinafter (esterification, azide substitution, polymerization):

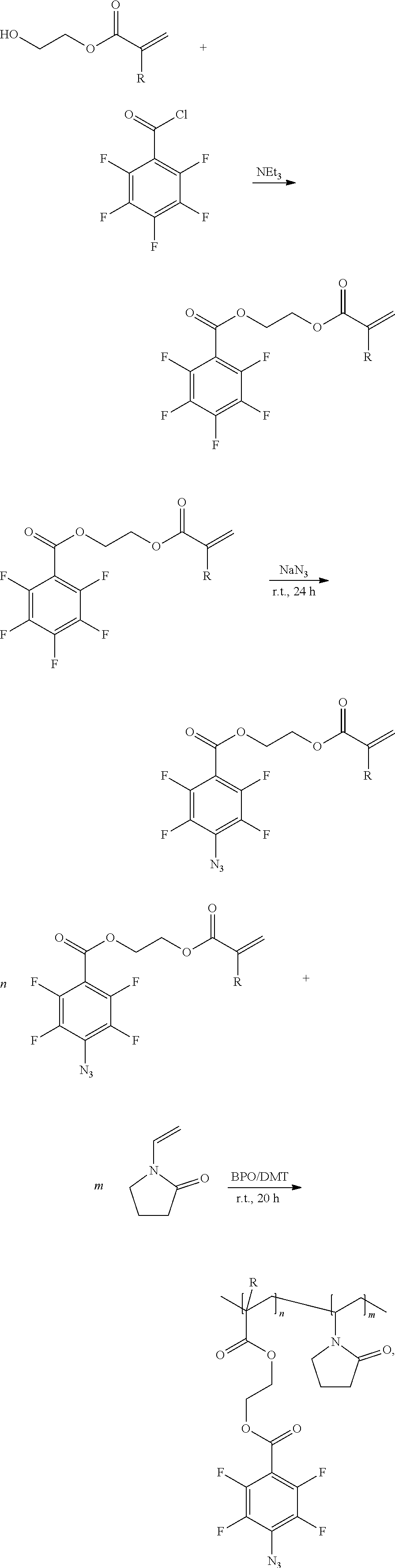

wherein n is an integer of 1, 2 or 3, preferably 1 or 2, most preferably 1 and designates the number of perfluorophenylazide units, specifically the perfluorobenzoyl (meth)acrylate azide units, and m is an integer in the range of from 15 to 35, preferably 20 to 30, which designates the number of vinylpyrrolidone units, and wherein R is H or CH₃.

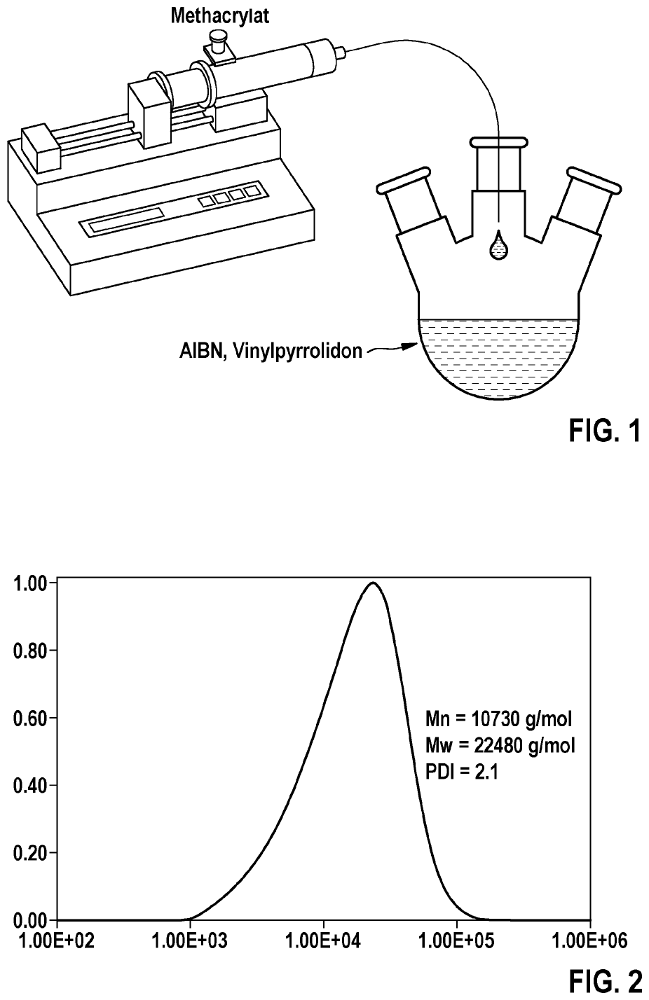

The polymerization can be optimized by adding the azidoperfluorophenyl (meth)acrylate, for example, via a syringe pump and/or over a period of 4 to 8 hours. The addition over a period of 4 to 8 hours of the acrylate promotes the formation of a copolymer with statistic distribution of the repeating units, due to the different copolymerization parameters of the monomers used, such as their different reaction rates. Contrary, a fast addition (less than 4 hours) and/or an addition without the syringe pump of the acrylate promotes the formation of a blockcopolymers. As an example, for methacrylate r=approx. 3.9, and for vinylpyrrolidone r=0.004. FIG. 1 shows an exemplary procedure. According to FIG. 1, the azidoperfluorophenyl (meth)acrylate is slowly added (via the syringe pump), e.g. over a time period of 4 to 8 hours, into the polymerization vessel containing vinylpyrrolidone and an initiator (e.g. AIBN as shown).

For an exemplary copolymer of the invention, FIG. 2 shows the molecular weight distribution by means of GPC (gel permeation chromatography) in units of normalized intensity vs. molar mass (g/mol). A unimodal distribution is obtained.

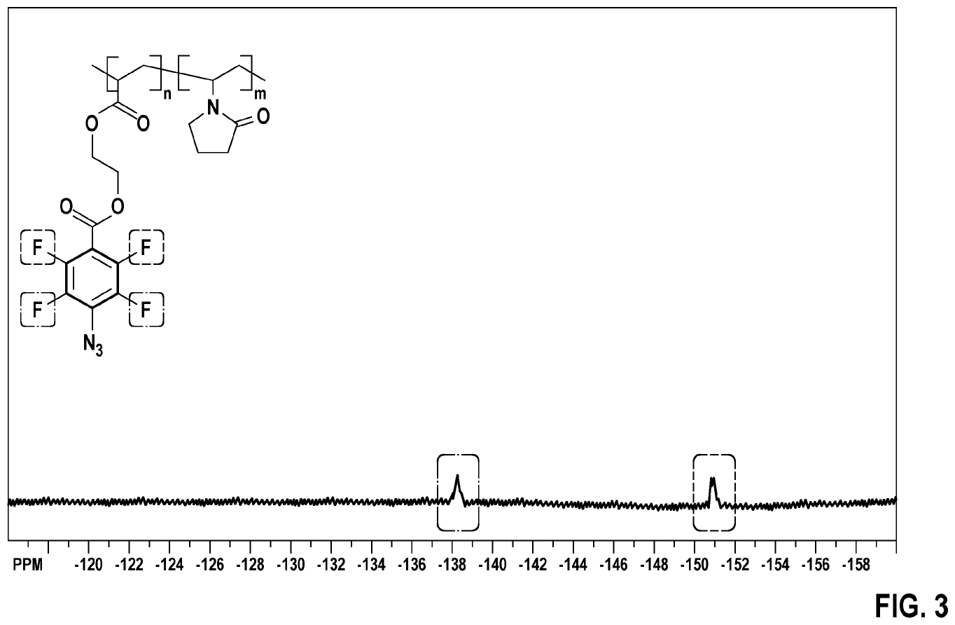

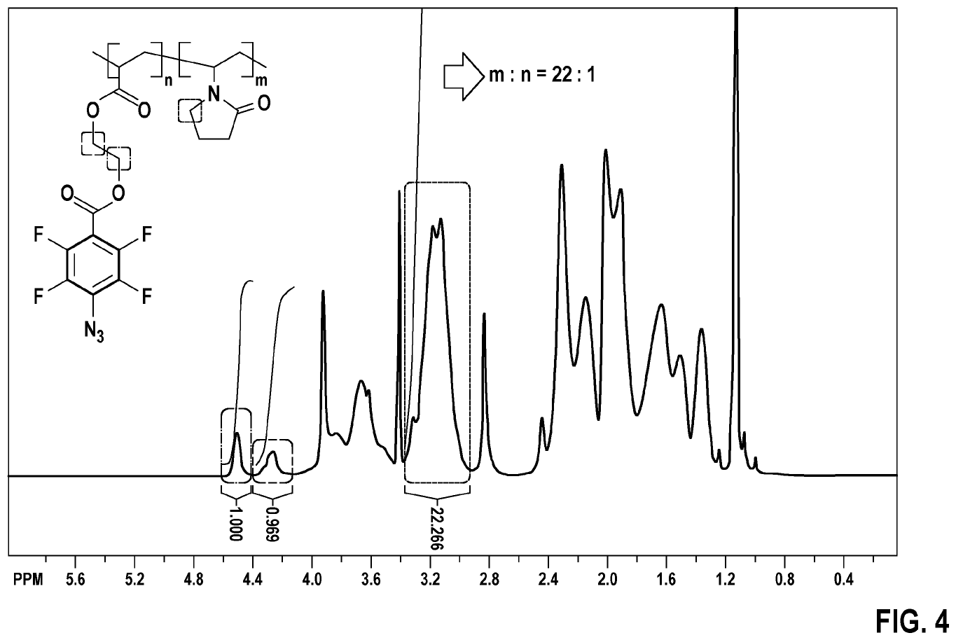

FIGS. 3 and 4 show ¹⁹F-NMR and ¹H-NMR spectra. FIG. 4 also shows how the ratio of the two repeating units to each other or the two parameters m and n can be determined from the ¹H-NMR spectrum due to H atoms at certain positions.

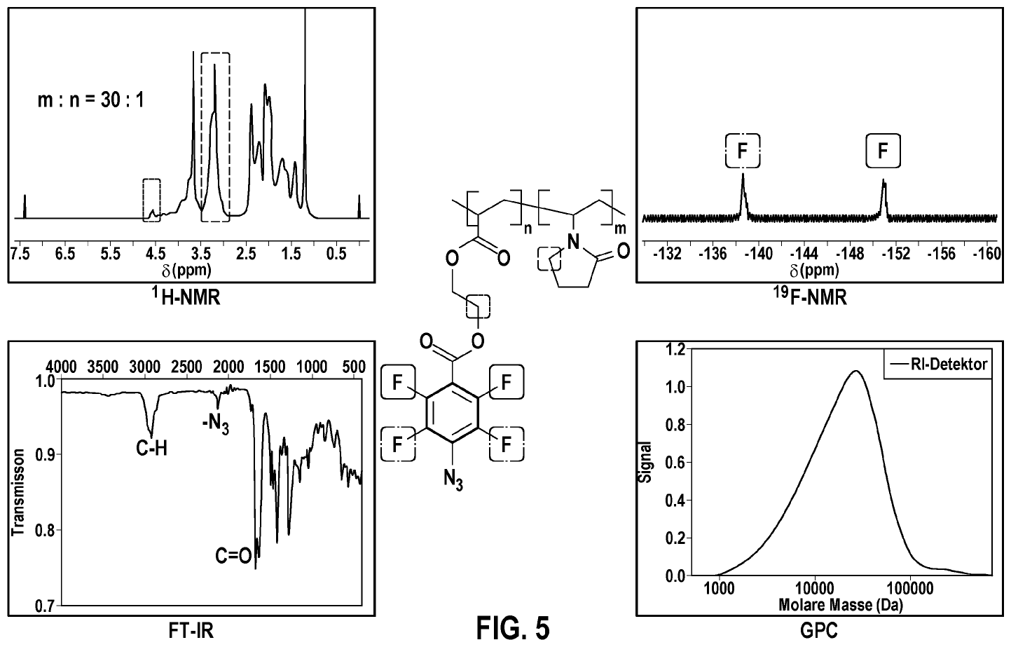

The ¹H-NMR, ¹⁹F-NMR and FT-IR spectra (transmission vs. wavenumber (cm⁻¹)) as well as molecular weight distribution for another exemplary copolymer are shown in FIG. 5.

In exemplary embodiments, the copolymerization is carried out using dibenzoyl peroxide (BPO) in N,N-Dimethyl-p-toluidine (DMT) at room temperature. A suitable reaction scheme is shown hereinafter:

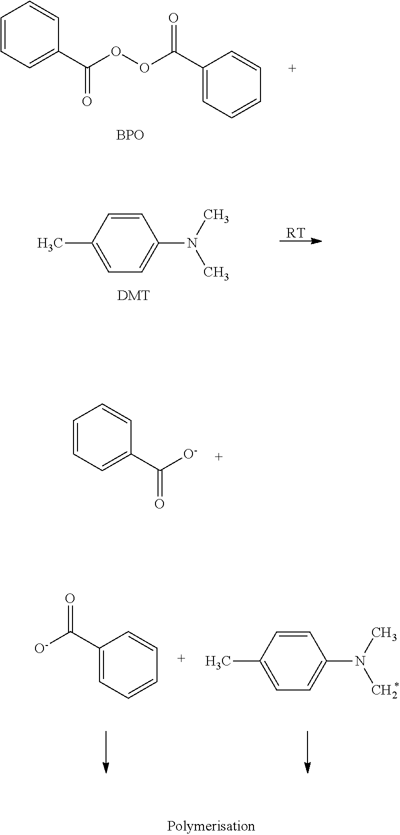

Alternatively, the copolymerization can be carried out using reversible-addition-fragmentation chain-transfer (RAFT) polymerization in the presence of BPO, DMT and benzyl 1H-imidazole-1-carbodithioate (BICDT). A suitable reaction is shown hereinafter:

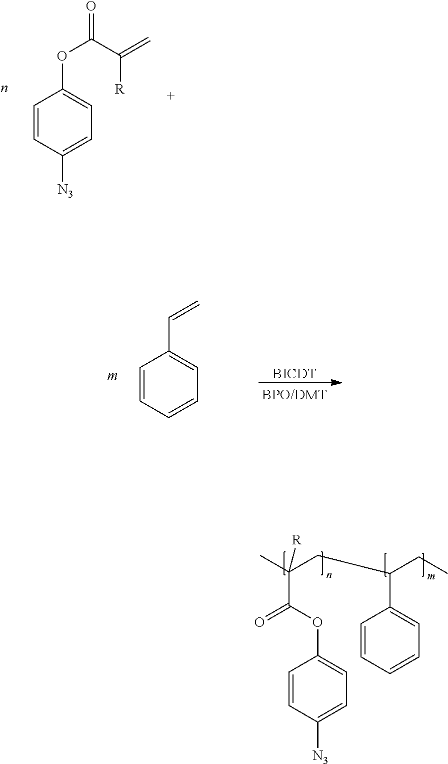

wherein R is H or CH₃.

It is also possible to change in the described preparation method with the steps (a), (b) and (c), the order of the steps (b) and (c), that is, the copolymerization is carried out before the azide substitution. An exemplary reaction route is shown hereinafter (top: free radical polymerization; bottom: azide substitution):

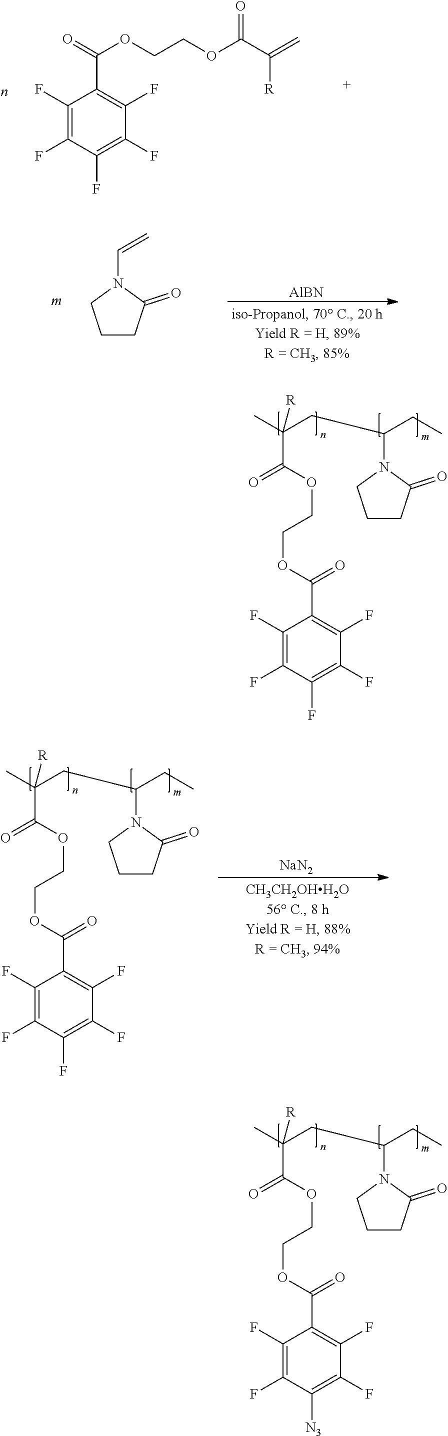

In further preferred embodiments of the invention, the copolymerization in step (c) is carried out in the presence of further monomers, for example, 2-hydroxyethyl methacrylate (HEMA), N,N-dimethyl (meth)acrylate, and N,N-dimethylacrylamide (DMA). Exemplary repeating units are shown hereinafter:

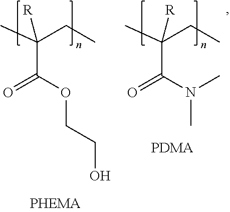

wherein R is H or CH₃. PHEMA denotes poly (2-hydroxyethyl methacrylate) (HEMA) and PDMA denotes poly (N,N-dimethylacrylamide).

Exemplary copolymers include at least one of the following repeating units:

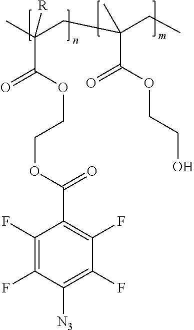

wherein R is H or CH₃ and wherein, for example, the molecular weight Mn is 33,000 g/mol the ratio min is 20:1 and preferably the PFPA content is 5 mol %. Preferably, the copoylmer is PHEMA-co-PFPA having a molecular weight Mn of 33,000 g/mol; the ratio m:n is 20:1; the PFPA content is 5 mol %. The solvent is preferably ethanol/water in a ratio of 1:1; and the concentration of the copolymer is 20 weight %.

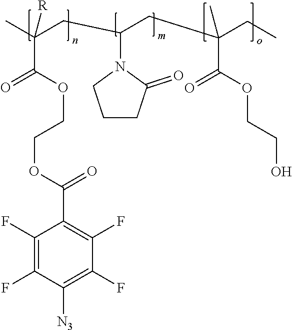

wherein R is H or CH₃ and wherein, for example, the molecular weight Mn is approx. 50,000 g/mol; the ratio m:o:n is 20:10:1 and wherein preferably the PFPA content is 3 mol % and the PDMA content is 32 mol %. Preferably, the copolymer is PVP-co-PHEMA-co-PFPA wherein the PFPA content is 3 mol %; and the PDMA content is 32 mol %.

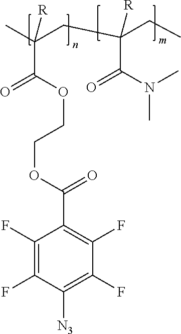

wherein, for example, the molecular weight Mn is 14,000 g/mol; the ratio m:n is 20:1 wherein preferably the PFPA content is 5 mol %. Preferably, the copolymer is PDMA-co-PFPA wherein the PFPA content is 5 mol %. The solvent is preferably acetone/water in a ratio of 1:1; and the copolymer concentration is 20 weight %.

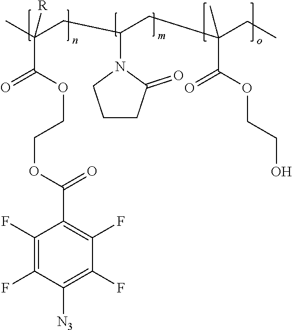

wherein R is H or CH₃ and wherein, for example, the molecular weight Mn, in g/mol, is 12,000 to 30,000; the ratio m:o:n is 20:1:1 to 20:10:1 and preferably the PFPA content is 3 mol % to 5 mol % and preferably the PDMA content is 5 mol % to 32 mol %. Preferably, the copolymer is PVP-co-PDMA-co-PFPA having a molecular weight Mn, in g/mol, of 30,000 or 12,000; the ratio m:o:n is 20:1:1 or 20:10:1; and preferably the PFPA content is 5 mol % or 3 mol %; and the PDMA content is 5 mol % or 32 mol %. The solvent is preferably acetone/water in a ratio of 1:1; and the copolymer concentration is 20 weight %.

The present invention further provides use of a coating composition for coating a surface of a vascular medical product, wherein the coating composition includes:
a) 0.1 to 10 weight % of polyvinylpyrrolidone having a molecular weight of less than 1,000,000 g/mol,
b) 0.1 to 10 weight % of polyvinylpyrrolidone having a molecular weight of more than 1,000,000 g/mol,
c) 0.1 to 5 weight % of a copolymer of the invention, and
d) 0 to 1 weight % of a multifunctional UV-sensitive lower-molecular phenyl azide compound having an UV-absorption, preferably in the range of 200 to 300 nm.

The components a) to d) are dissolved in aqueous alcohol, preferably in 50 to 90 volume % of alcohol, and 10 to 50 volume % of water. The alcohol is preferably ethanol and/or 2-propanol.

The copolymer c) is a copolymer as described above.

The phenyl azide compound d) is preferably a triazide, polyethyleneglycol-based bisazide, diol bisazide, and/or an ionic bisazide. Exemplary multifunctional phenyl azide compounds are shown hereinafter (in this order: triazide, PEG-bisazide, diol-bisazide, ionic bisazide):

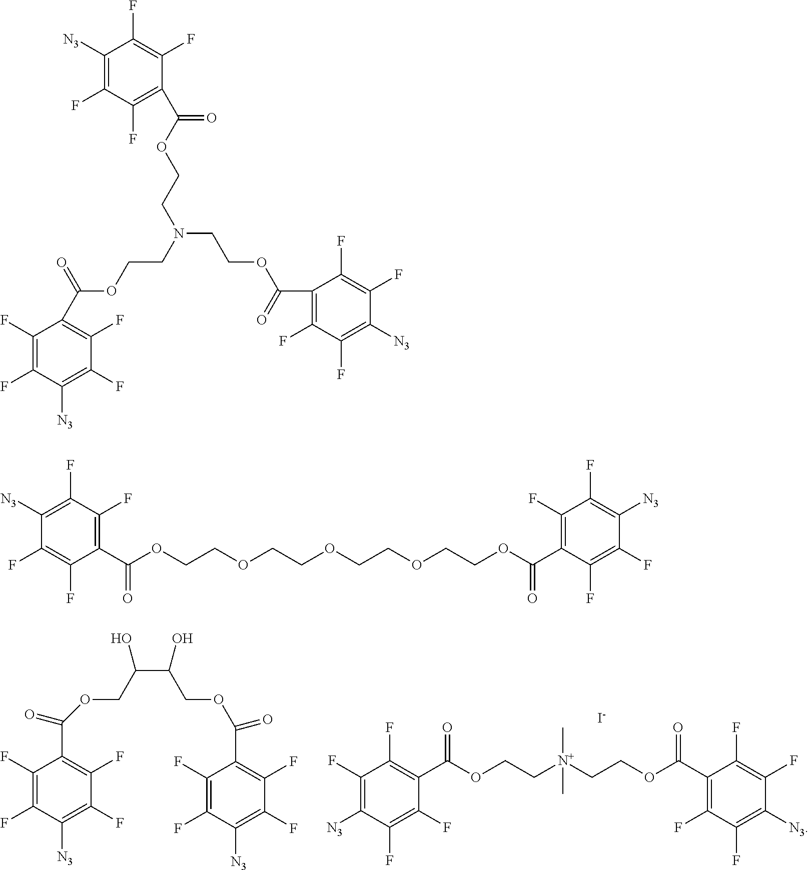

The present invention further provides a method for coating a surface of a vascular medical product. In this method, a coating composition of the invention or a coating composition as described above, is applied to the surface of a vascular medical product by a procedure known in the art. Suitable application methods are dip coating, spray coating, roller coating, printing, or painting. The coating composition also can be applied by means of a doctor blade, a doctor roller, or Langmuir-Blodgett films. A preferred method is dip-coating. After its application on the surface, the applied composition is dried. Thereafter, the composition is irradiated by UV light. The UV light has preferably a wavelength in the range of from 200 to 350 nm, more preferably from 240 and 320 nm. A suitable wavelength is, for example, 254 nm or 265 nm, e.g., by using a Hg lamp.

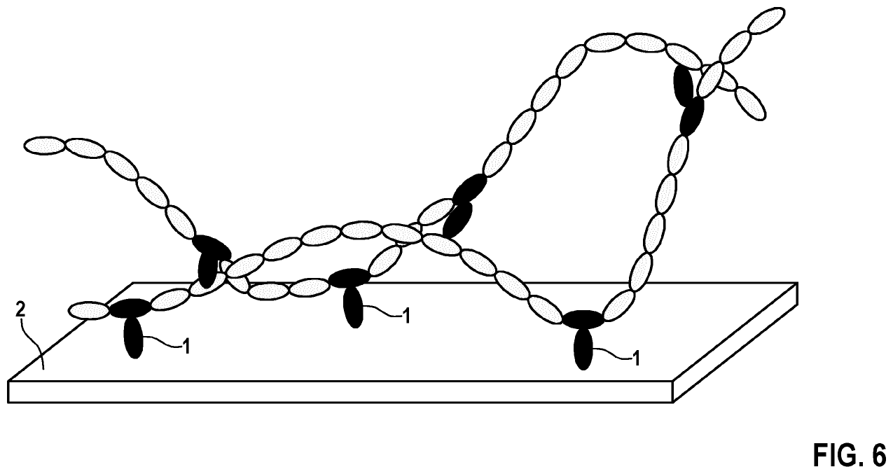

FIG. 6 shows schematically the attachment of the copolymer to the substrate. In FIG. 6, the adhesion points 1 to the substrate 2 surface represent the perfluoro groups of the copolymer. The points shown as being not attached to the surface represent hydrophilic groups, such as polyvinylpyrrolidone units. The covalent binding of the copolymer to the surface (after UV radiation) can be analyzed, for example, by FT-IR through the disappearance of the azide peak, said peak appearing at 2134 cm⁻¹ in the spectrum before UV radiation.

In a preferred embodiment, the substrate is dipped into the coating composition and drawn out from the coating composition at a defined velocity to obtain a defined coating thickness. The film thickness of the coating can be determined by the Landau-Levich equation:

$$h = 0.94 \frac{{( {v}_{0} η )}^{2 / 3}}{{( g ρ )}^{1 / 2} {γ}_{LV}^{1 / 6}}$$

wherein ν is the velocity, η is the viscosity, ρ is the density, and γ is the surface tension. The film thickness can be e.g., 2 μm.

The substrate of the vascular medical product can include a polymer selected from polyamide, for example, polyamide 11 or polyamide 12; polyetherblockamides; thermoplastic polyurethanes (TPU); polyester, for example, PET, or polyester-based elastomers; polyolefins, for example, HDPE, LDPE, PP or olefin-based co-polymers; vinylpolymers, for example, polystyrene; polysiloxanes; and combinations of one or more thereof.

In preferred embodiments of the invention, the vascular medical product is a medical product or part thereof, which is intended to be implanted or introduced into vessels, particularly in the fields of electrophysiology, heart-rhythm management, neurostimulation, vascular and endovascular intervention, intervention in and at the heart. Particularly preferred vascular medical products are selected from coronary or peripheral catheters or parts thereof, CRM devices or parts, balloon parts, or pacemaker electrodes.

In another preferred embodiment, the coating method of the invention is carried out in automated manner.

The present invention further provides a coated vascular medical product that is obtained by the coating according to the invention or the coating method described above.

The coatings obtained by the invention exhibit low friction to friction partners, e.g., blood vessels, and release of a very low number of particles. The release of particles can be determined by a particle counter using e.g., a simulated use model according to ASTM F2394-07 (2017), X.2.4. In an exemplary embodiment for a catheter with hydrophilic coated particles, a desirable value is less than 2000 particles release, wherein the limit for particles above 10 μm is 6000 particles. The friction of the coating scan be determined by the friction coefficient COF.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A method for coating a surface of a vascular medical product or at least one part thereof, comprising:
prep ring a copolymeric coating comprising or consisting of repeating units of a) vinylpyrrolidone and b) at least one perfluorophenyl azide moiety; and
applying the copolymeric coating to the vascular medical product or the at least one part thereof.

2. The method according to claim 1, wherein the applying comprises one or more of dip coating, spray coating, roller coating, printing, painting, or application with a doctor blade, doctor roller or Langmuir-Blodgett films.

3. The method according to claim 2, wherein the applying is via a copolymeric coating solution.

4. The method according to claim 3, wherein the copolymer coating solution comprises the copolymer dissolved in 50 to 90 volume % of alcohol.

5. The method according to claim 1, wherein the surface of the vascular medical product comprises or is made of a polymer selected from polyamide, polyetherblockamides, thermoplastic polyurethanes (TPU), polyester, polyester-based elastomers, thermoplastic elastomers, polyolefins, vinylpolymers, polysiloxanes, and combinations thereof.

6. The method according to claim 1, wherein the copolymer comprises 1 to 50 mol % of the repeating unit a), and 0.1 to 5 mol % of a repeating unit b).

7. The method according to claim 1, wherein the copolymeric coating comprises:
i) 0.1 to 10 weight % of polyvinylpyrrolidone having a molecular weight of less than 1,000,000 g/mol,
ii) 0.1 to 10 weight % of polyvinylpyrrolidone having a molecular weight of more than 1,000,000 g/mol,
iii) more than 0 weight % and up to 6 weight % of the perfluorophenyl azide.

8. The method according to claim 7, wherein more than 0 weight % and up to 0.3 weight % are of the perfluorophenyl azide.

9. The method according to any claim 1, wherein the copolymer further comprises at least one repeating unit c) selected from of 2-hydroxyethyl methacrylate), N,N-dimethyl (meth)acrylate, and N,N-dimethylacrylamide.

10. The method according to claim 9, wherein the copolymer comprises 1 to 20 mol % of the repeating unit c).

11. The method according to claim 1, wherein the copolymer has the following chemical structure: 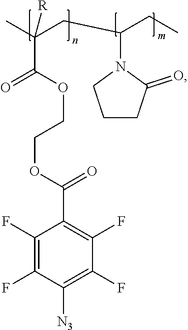
wherein R is H or CH₃ wherein n is an integer 1 or 2, and m is an integer in the range of from 15 to 35; or 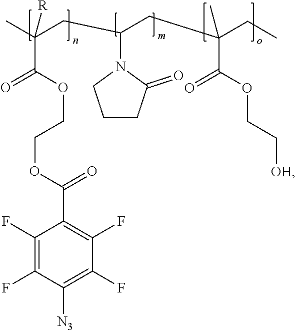
wherein n is 1 or 2, m is 15 to 25, and o is 5 to 15; 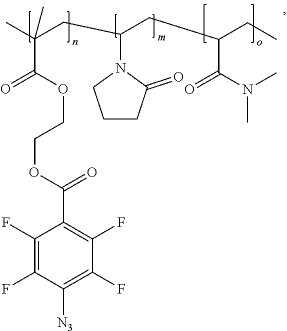
wherein n is 1 or 2, m is 15 to 25, and o is 5 to 15.

12. A vascular medical product or at least one part thereof, comprising a copolymeric coating, wherein the copolymeric coating is bound to a surface of the vascular medical product and comprises at least one of the following copolymers or consists of one of the following copolymers:

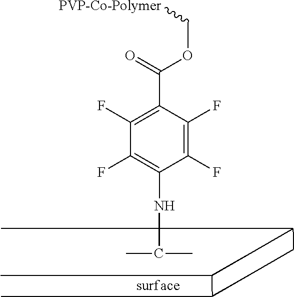

13. The vascular medical product or the at least one part thereof according to claim 12, wherein the copolymeric coating comprises at least one of the following copolymers or consists of one of the following copolymers: 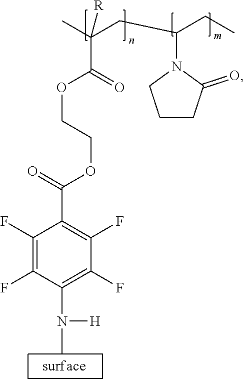
wherein R is H or CH₃ and wherein n is an integer 1 or 2, and m is an integer in the range of from 15 to 35; or 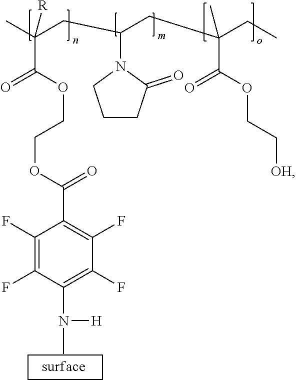
wherein R is H or CH₃ and wherein n is 1 or 2, m is 15 to 25, and o is 5 to 15; or 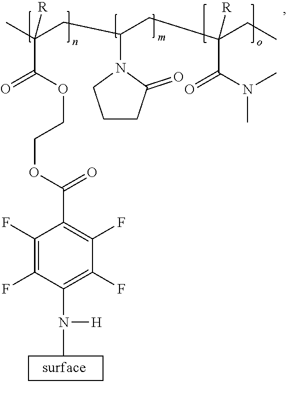
wherein R is H or CH₃ and wherein n is 1 or 2, m is 15 to 25, and o is 5 to 15.

14. The vascular medical product or the at least one part thereof of claim 12, wherein the vascular medical product or the at least one part thereof is configured to be implanted or introduced into a vessel.

15. The vascular medical product or the at least one part thereof of one of claim 12, wherein the surface of the vascular medical product comprises or is made of a polymer selected from polyamide, polyetherblockamides, thermoplastic polyurethanes (TPU), polyester, polyester-based elastomers, thermoplastic elastomers, polyolefins, vinylpolymers, polysiloxanes, and combinations thereof.

16. A vascular medical product or at least one part thereof, comprising a copolymeric coating that comprises or consists of a copolymer having at least
a) repeating units of vinylpyrrolidone, and
b) at least one perfluorophenyl azide moiety.

17. The vascular medical product or at least one part thereof of claim 16, wherein the copolymer comprises repeating units selected from of 2-hydroxyethyl methacrylate, N,N-dimethyl (meth)acrylate, and N,N-dimethylacrylamide.

18. The method of claim 1, comprising drying the copolymeric coating.

19. The method of claim 1, comprising irradiating the copolymeric coating UV irradiation.
